(19) United States
(12) Patent Application Publication
(10) Pub. No.: US 2007/0073303 A1
(43) Pub. Date: Mar. 29, 2007

(54) Bone-cutting circular saw

(76) Inventor: Robert S. Namba, Corona del Mar, CA (US)

Correspondence Address: GIFFORD, KRASS, GROH, SPRINKLE & CITKOWSKI, P.C, PO BOX 7021, TROY, MI, 48007-7021, US

(21) Appl. No.: 11/223,683

(22) Filed: Sep. 9, 2005

(51) Int. Cl. A61B 17/00

(52) U.S. Cl. 606/82

(57) ABSTRACT

A power bone-cutting saw system exhibits the cutting efficiency of a circular saw, but retains the practical dimensions of an oscillating saw blade. A circular saw blade, available in a variety of diameters, is positioned at the end of a narrow, low-profile elongated support. The support houses a drive assembly that efficiently transmits power from a standard hand-held portable saw to the blade. The support can rest on modified saw guides for total joint procedures, providing the surgeon with a compact, efficient and precise bone-cutting tool. In the preferred embodiment the circular saw and support are single-use disposable units, available in a variety of lengths and widths. In an alternative embodiment, twin blades are used to eliminate changes in height between the blades and the support.

### FIELD OF THE INVENTION

The present invention relates generally to surgical saws and, more particularly, to powered circular saws and blades for precision cutting of bone.

### BACKGROUND OF THE INVENTION

Powered surgical saws are utilized in many operations in orthopedic surgery, especially during total-joint replacement procedures. For total-knee replacement surgery, accurate bone cuts are required to ensure optimal implant alignment to maximize durability and function of the artificial joint.

Conventional total-knee replacement tools include cutting blocks (cutting guides) containing a slot and powered oscillating saws. The slot of the cutting blocks permit passage of the oscillating saw blade, thereby guiding the angle and position of the intended bone cut.

Oscillating saws are utilized to cut bone in many surgical procedures, including total-knee replacement and total-hip replacement surgery. The saw blades attached to oscillating saws are long and narrow, allowing the surgeon to cut thick pieces of bone with the limited exposure offered with routine surgical approaches.

Oscillating saws are inherently inefficient cutting tools, however, requiring considerable manual force to cut hard materials such as bone. The saw blades vibrate, whip and deflect, leading to gouging of bone, and imprecise cuts. Though the cutting teeth must oscillate to cut bone, oscillation of the shank of the saw blade can damage soft tissues, particularly with minimally invasive surgical approaches.

The typical length of oscillating saw blades are about 3½ inches in length, limited by deflection of the blade beyond this length. However, the combination of the cutting guide and bone may be thicker than 3½ inches. Metallic debris is often generated from the oscillating blade scraping on the cutting block. Vibration of the blade on the cutting block can loosen or shift the cutting block, leading to excess bone removal. Binding of the saw blade within bone can result in kickback, potentially injuring vital structures such as ligaments, tendons, nerves and blood vessels. In addition, the deflected saw blade can injure the surgeon or assistant's hands, exposing them to possible blood-borne pathogens.

To alleviate these problems, alternative bone-cutting systems have been developed. As one example, U.S. Pat. No. 5,725,530 describes a surgical saw including a saw assembly driven by a powered surgical handpiece. The system includes a pair of parallel, co-planar guide arms, a pair of flexible, endless cutting bands disposed around the guide arms, respectively, and a drive mechanism for driving the cutting bands around the guide arms in a cutting direction. The cutting bands each include a plurality of spaced cutting teeth connected to one another by flexible band segments. The cutting bands are driven by the drive mechanism relative to the guide arms in opposite directions along defined paths to cut anatomical tissue at distal ends of the guide arms. A method of resecting bone includes the steps of driving the cutting bands relative to the guide arms in the cutting direction along the defined paths and inserting the distal ends of the guide arms in the bone to resect the bone along the plane of the guide arms.

Although systems such as the one just described do away with an oscillating blade, the assembly is complex, leading to increased cost or possible mechanical problems. Circular saws are efficient cutting tools which afford great precision in cutting hard substances. The drawbacks of circular saw blades are that they have limited travel, practical only for cutting relatively thin structures, and have a broad cutting base, requiring exposure of a large segment of the substance being cut.

### SUMMARY OF THE INVENTION

This invention resides in a power cutting saw system affording greater efficiency and improved precision in cutting bone. Broadly, the system exhibits the cutting efficiency of a circular saw, but retains the practical dimensions of an oscillating saw blade.

According to the invention, a circular saw blade, available in a variety of diameters, is positioned at the end of a narrow, low-profile elongated support. The support houses a drive assembly that efficiently transmits power from a standard hand-held portable saw to the blade. The support can rest on modified saw guides for total joint procedures, providing the surgeon with a compact, efficient and precise bone-cutting tool.

In the preferred embodiment the circular saw and support are single-use disposable units, available in a variety of lengths and widths. In an alternative embodiment, twin blades are used to eliminate changes in height between the blades and the support.

### BRIEF DESCRIPTION OF THE DRAWINGS

FIG. 1 is an oblique view of a circular saw and support according to the invention;

FIG. 2A is a view of a support and smaller saw blade;

FIG. 2B is a drawing of a support and larger saw blade;

FIG. 3 is a side view of a saw according to the invention attached to a hand-held portable drive;

FIG. 4 is a side view of a saw and support providing additional detail;

FIG. 5A is an exploded-view drawing of an alternative embodiment of the invention incorporating twin blades to eliminate changes in height along the length of the tool; and

FIG. 5B is an assembled view of the embodiment of FIG. 5A.

### DETAILED DESCRIPTION OF THE INVENTION

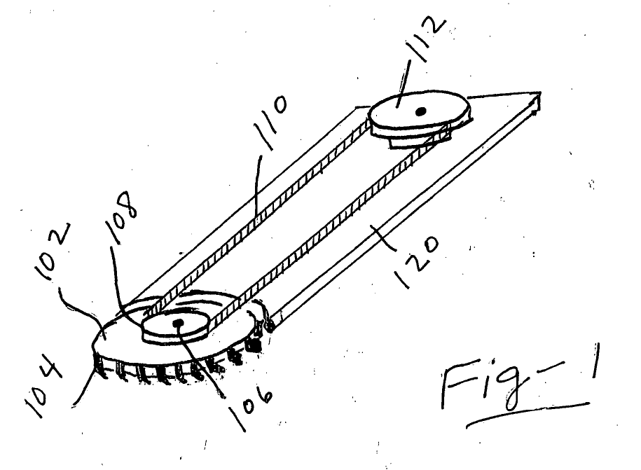

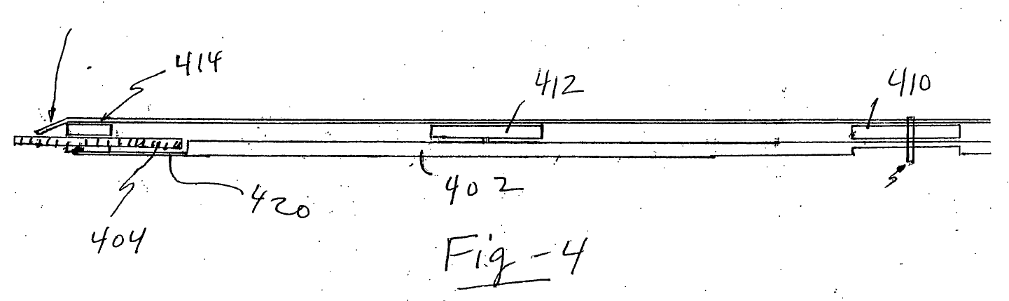

Turning now to the figures, FIG. 1 is an oblique representation of a preferred embodiment of the invention, including a saw blade 102 having teeth 104 rotatable about an axis 106. A belt 110 is dressed around a raised circular boss 106 on the blade 102. The belt 110 is driven from a pulley 112, which in turn, is driven by a motor disposed in hand-held tool, illustrated in FIG. 3.

Although in the preferred embodiment the saw blade is belt-driven, other mechanisms may be used, including meshing gears, as well as direct drive to the teeth 104 of the blade 102. With respect to the teeth 104, any configuration suitable for bone cutting may be utilized, as the invention is not limited in this regard. For example, the teeth currently used on oscillating saws may be applicable, as well as other configurations known to those of skill in the art.

In terms of materials, the blade 102 and support 120 are made of any suitable durable and rigid material, such as metal. Nor is the invention limited in this regard, since it may be possible to use hard plastics for various components as well. This may be advantageous for single-use disposable versions of the invention.

Although the top of the assembly includes a slight stair-step in terms of height between the blade 102 and the pulley 108, the bottom of the assembly is flat, allowing the surgeon to rest the support 120 onto existing or modified saw guides for various surgical procedures, including total-joint replacement. Little vibration occurs between the novel circular saw/support and the cutting guide since only the exposed tip of the circular saw moves. Since the platform does not vibrate on the cutting block, there is less metallic debris formed, and less deviation from the intended course. Precise bone cuts are thereby facilitated, providing a safe and stable cutting instrument for the surgeon.

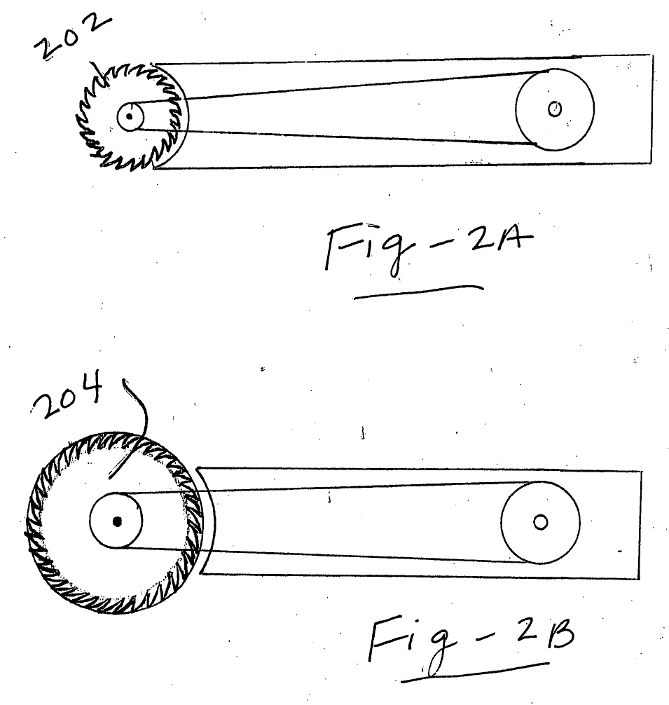

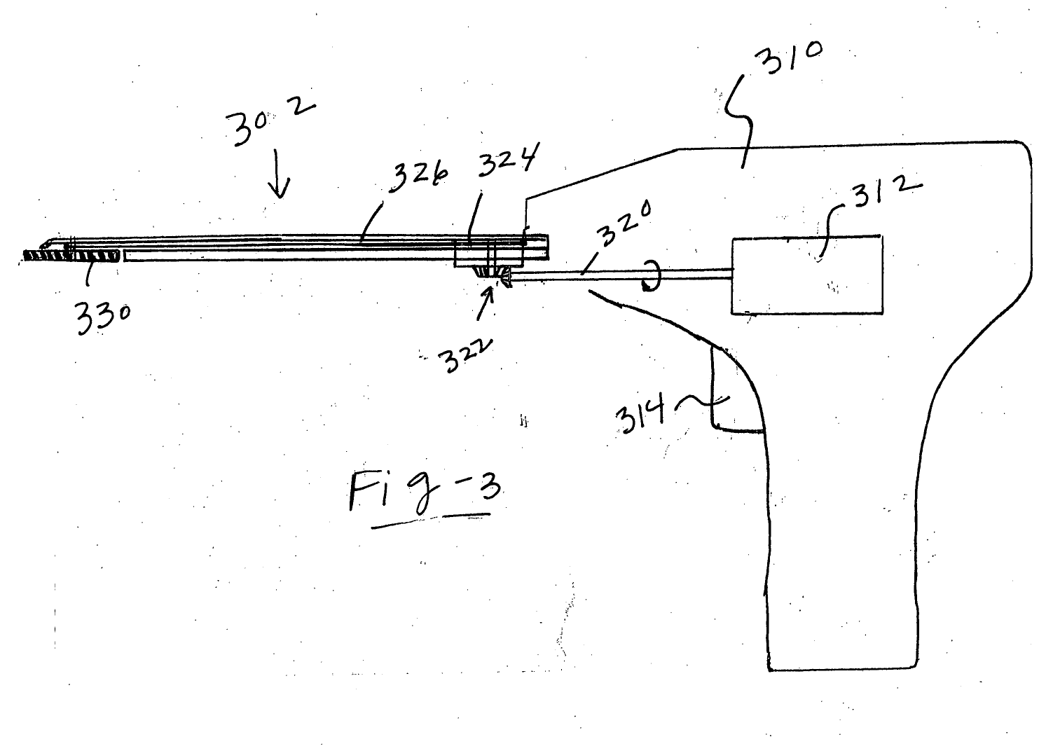

It is anticipated that different assemblies of the type shown in FIG. 1 will be provided, with different-diameter blades and/or teeth configurations. For example, FIG. 2A shows an embodiment utilizing a relatively small blade 202, whereas FIG. 2B shows an embodiment with a larger blade 204. Dimensionally the diameter of the blade is in the range of 1 to 2 inches, more or less, and the length of the support and blade is preferably 6 inches or less. The width is preferably sufficient to fit into existing cutting guide though custom guides may be provided if this is not possible.

FIG. 3 is a side-view drawing showing an assembly 302 according to the invention mounted on a hand-held drive 310 including motor 312 and finger control 314. Although the inventive blade and platform may be provided in combination with the hand-held drive 310, the blade and platform combination in and of itself is believed to be patentably distinct, enabling it to be attached and removed from an appropriate hand-held drive. In FIG. 3, the motor 312 turns a shaft 320 which, in turn, transmits power through bevel gears 322 to a pulley 324, belt 326 and blade 330.

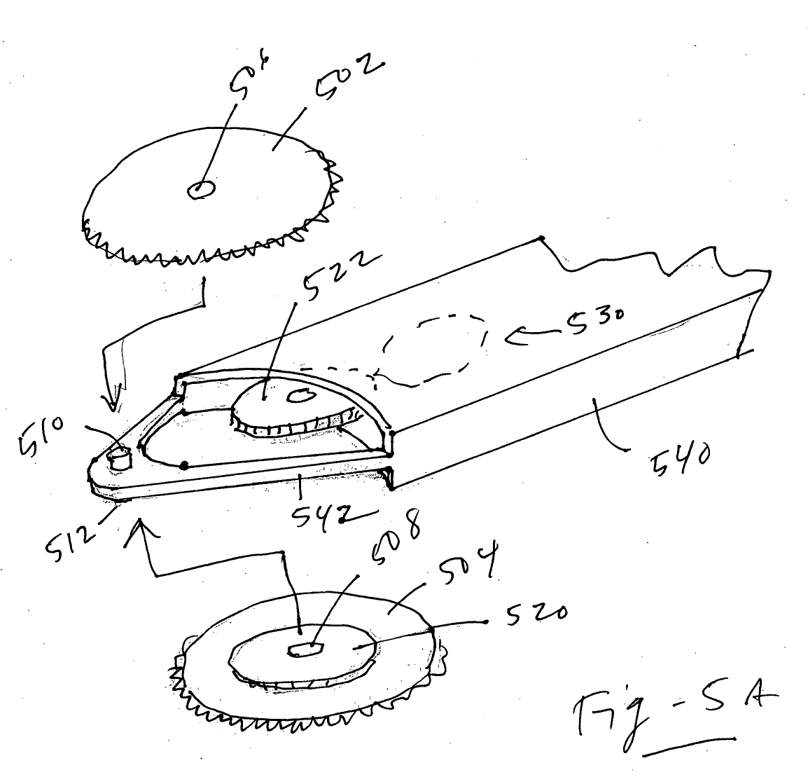

FIG. 4 is a more detailed drawing of a support 402 onto which there is mounted a circular saw blade 404. Although the belt drive is not shown, pulleys 410, 412, 414 are shown, with pulley 414 being an optional intermediate pulley to maintain tension and precision. The blade 404 is pivotally attached to the support 402 through an extension 420. Note that a top cover, 422, includes a bent portion forming a beveled front edge to better facilitate plunge-type operations. The beveled front end acts as a wedge, such that as the blade is driven into bone, the beveled portion deflects unwanted bone and other fragments away from the support itself, keeping the area free of debris.

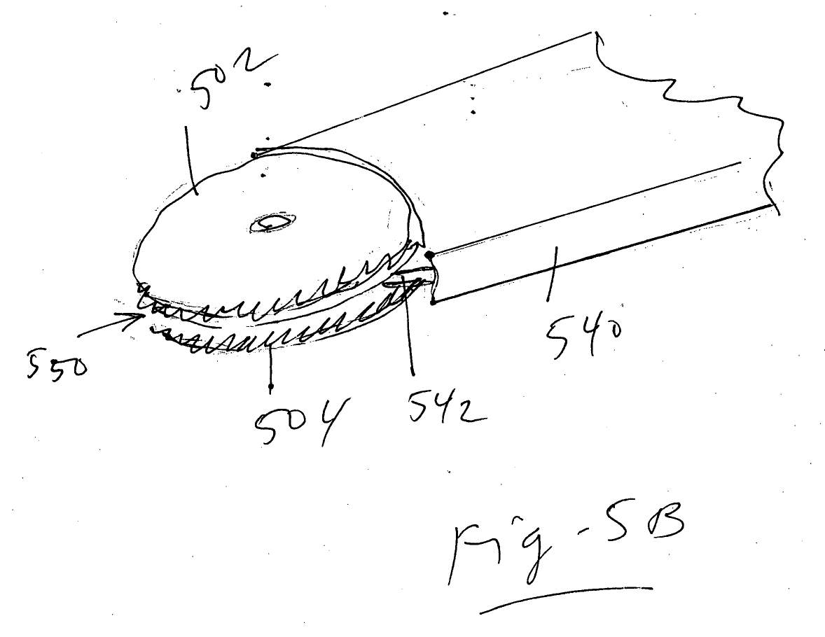

FIGS. 5A and B show a twin-blade embodiment of the invention, which avoids stair-steps in terms of height, thereby eliminating the need for a beveled front edge. In this embodiment, a top blade 502 and a bottom blade 504, having apertures 506, 508, respectively, fit onto pins 510, 512 on the support arm 542 from a housing 540. The blades 502, 504 each include mechanisms for driving such as gears 520 (the gear associated with blade 502 is not visible in the drawing). These gears mesh with a gear 522 within the housing 540, which is, in turn, driven by a belt, gear 530, or other suitable drive means. Again, depending upon the tooth configuration of the blades, the teeth may also function as gears. FIG. 5B is an oblique drawing of the assembled embodiment of FIG. 5A. Although a slight gap 550 exists between the two blades, given the fragile nature of bone having such dimensions, it easily breaks away, allowing for a plunge-type cut without any height interference.

I claim:

1. A surgical saw, comprising:
an elongated support having proximal and distal ends and a width;
a circular saw blade suitable for bone cutting rotationally mounted on the distal end;
an interconnection to a source of mechanical power disposed on the proximal end; and
a drive mechanism between the interconnection and the circular saw blade, such that when the support is interconnected to a source of mechanical power, the blade rotates for bone-cutting purposes.

2. The surgical saw of claim 1, wherein:
the interconnection to a source of mechanical power includes a pulley; and
the drive mechanism includes a belt from the pulley to the saw blade.

3. The surgical saw of claim 1, wherein:
the saw blade has a diameter which equal to or greater than the width of the support.

4. The surgical saw of claim 1, wherein the support has a flat bottom surface adapted to rest against the edge of a cutting guide.

5. The surgical saw of claim 1, including two saw blades, both disposed at the distal end of the support.
